(19) United States
(12) Patent Application Publication
(10) Pub. No.: US 2010/0256752 A1
(43) Pub. Date: Oct. 7, 2010

(54) PROSTHETIC HEART VALVES, SUPPORT STRUCTURES AND SYSTEMS AND METHODS FOR IMPLANTING THE SAME,

(76) Inventors: David C. Forster, Los Altos, CA (US); Brian Beckey, Woodside, CA (US); Scott Heneveld, Whitmore, CA (US); Brandon Walsh, Syracuse, UT (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 12/439,720

(22) PCT Filed: Sep. 6, 2007
(86) PCT No.: PCT/US07/77741

§ 371 Date: Jun. 18, 2010

Related U.S. Application Data
(60) Provisional application No. 60/824,732, filed on Sep. 6, 2006.

(51) Int. Cl. A61F 2/24

(52) U.S. Cl. 623/2.17

(57) ABSTRACT

The implantable heart valve can have an inverted configuration and can include a support structure and at least two valve leaflets. Preferably, when the valve is placed in proximity with an aortic valve implantation site of a subject and engaged with the implantation site, the valve leaflets are configured to deflect towards the aortic wall into a first position for resisting the flow of blood towards the left ventricle and configured to deflect away from a aortic wall into a second position for allowing the flow of blood from a left ventricle, with the support structure configured to remain static during leaflet deflection.

### FIELD OF THE INVENTION

The present invention relates generally to medical devices and methods. More particularly, the present invention relates to prosthetic heart valves, structures for providing scaffolding of body lumens, and devices and methods for delivering and deploying these valves and structures.

### BACKGROUND INFORMATION

Diseases and other disorders of the heart valve affect the proper flow of blood from the heart. Two categories of heart valve disease are stenosis and incompetence. Stenosis refers to a failure of the valve to open fully, due to stiffened valve tissue. Incompetence refers to valves that cause inefficient blood circulation by permitting backflow of blood in the heart.

Medication may be used to treat some heart valve disorders, but many cases require replacement of the native valve with a prosthetic heart valve. Prosthetic heart valves can be used to replace any of the native heart valves (aortic, mitral, tricuspid or pulmonary), although repair or replacement of the aortic or mitral valves is most common because they reside in the left side of the heart where pressures are the greatest. Two primary types of prosthetic heart valves are commonly used, mechanical heart valves and prosthetic tissue heart valves.

The caged ball design is one of the early mechanical heart valves. The caged ball design uses a small ball that is held in place by a welded metal cage. In the mid-1960s, another prosthetic valve was designed that used a tilting disc to better mimic the natural patterns of blood flow. The tilting-disc valves had a polymer disc held in place by two welded struts. The bileaflet valve was introduced in the late 1970s. It included two semicircular leaflets that pivot on hinges. The leaflets swing open completely, parallel to the direction of the blood flow. They do not close completely, which allows sonic backflow.

The main advantages of mechanical valves are their high durability. Mechanical heart valves are placed in young patients because they typically last for the lifetime of the patient. The main problem with all mechanical valves is the increased risk of blood clotting.

Prosthetic tissue valves include human tissue valves and animal tissue valves. Both types are often referred to as bioprosthetic valves. The design of bioprosthetic valves are closer to the design of the natural valve. Bioprosthetic valves do not require long-term anticoagulants, have better hemodynamics, do not cause damage to blood cells, and do not suffer from many of the structural problems experienced by the mechanical heart valves.

Human tissue valves include homografts, which are valves that are transplanted from another human being, and autografts, which are valves that are transplanted from one position to another within the same person.

Animal tissue valves are most often heart tissues recovered from animals. The recovered tissues are typically stiffened by a tanning solution, most often glutaraldehyde. The most commonly used animal tissues are porcine, bovine, and equine pericardial tissue.

The animal tissue valves are typically stented valves. Stentless valves are made by removing the entire aortic root and adjacent aorta as a block, usually from a pig. The coronary arteries are tied off, and the entire section is trimmed and then implanted into the patient.

A conventional heart valve replacement surgery involves accessing the heart in the patent's thoracic cavity through a longitudinal incision in the chest. For example, a median sternotomy requires cutting through the sternum and forcing the two opposing halves of the rib cage to be spread apart, allowing access to the thoracic cavity and heart within. The patient is then placed on cardiopulmonary bypass which involves stopping the heart to permit access to the internal chambers. Such open heart surgery is particularly invasive and involves a lengthy and difficult recovery period.

A less invasive approach to valve replacement is desired. The percutaneous implantation of a prosthetic valve is a preferred procedure because the operation is performed under local anesthesia, does not require cardiopulmonary bypass, and is less traumatic. Current attempts to provide such a device generally involve stent-like structures, which are very similar to those used in vascular stent procedures with the exception of being larger diameter as required for the aortic anatomy, as well as having leaflets attached to provide one way blood flow. These stent structures are radially contracted for delivery to the intended site, and then expanded/deployed to achieve a tubular structure in the annulus. The stent structure needs to provide two primary functions. First, the structure needs to provide adequate radial stiffness when in the expanded state. Radial stiffness is required to maintain the cylindrical shape of the structure, which assures the leaflets coapt properly. Proper leaflet coaption assures the edges of the leaflets mate properly, which is necessary for proper sealing without leaks. Radial stiffness also assures that there will be no paravalvular leakage, which is leaking between the valve and aorta interface, rather than through the leaflets. An additional need for radial stiffness is to provide sufficient interaction between the valve and native aortic wall that there will be no valve migration as the valve closes and holds full body blood pressure. This is a requirement that other vascular devices are not subjected to. The second primary function of the stent structure is the ability to he crimped to a reduced size for implantation.

Prior devices have utilized traditional stenting designs which are produced from tubing or wire wound structures. Although this type of design can provide for crimpability, it provides little radial stiffness. These devices are subject to “radial recoil” in that when the device is deployed, typically with balloon expansion, the final deployed diameter is smaller than the diameter the balloon and stent structure were expanded to. The recoil is due in part because of the stiffness mismatches between the device and the anatomical environment in which it is placed. These devices also commonly cause crushing, tearing, or other deformation to the valve leaflets during the contraction and expansion procedures. Other stenting designs have included spirally wound metallic sheets. This type of design provides high radial stiffness, yet crimping results in large material strains that can cause stress fractures and extremely large amounts of stored energy in the constrained state. Replacement heart valves are expected to survive for many years when implanted. A heart valve sees approximately 500,000,000 cycles over the course of 15 years. High stress states during crimping can reduce the fatigue life of the device. Still other devices have included tubing, wire wound structures, or spirally wound sheets formed of nitinol or other superelastic or shape memory material. These devices suffer from some of the same deficiencies as those described above.

### SUMMARY

Provided herein are implantable heart valves and methods for using the same. These valves and methods are provided by way of exemplary embodiments and in no way should be construed to limit the claims beyond the language that appears expressly therein.

In one exemplary embodiment, the implantable heart valve has an inverted configuration and includes a support structure and at least two valve leaflets. Preferably, when the valve is placed in proximity with an aortic valve implantation site of a subject and engaged with the implantation site, the valve leaflets are configured to deflect towards the aortic wall into a first position for resisting the flow of blood towards the left ventricle and configured to deflect away from a aortic wall into a second position for allowing the flow of blood from a left ventricle, with the support structure configured to remain static during leaflet deflection.

Other systems, methods, features and advantages will be or will become apparent to one with skill in the art upon examination of the following figures and detailed description. It is intended that all such additional systems, methods, features and advantages be included within this description, be within the scope of the systems and methods described herein, and be protected by the accompanying claims.

### BRIEF DESCRIPTION OF THE FIGURES

FIG. 1A is a longitudinal cross-sectional view depicting a native aortic valve within the heart.

FIG. 1B is a longitudinal cross-sectional view depicting the native aortic valve in the valve-closed position.

FIG. 1C is a radial cross-sectional view taken along line 1C-1C of FIG. 1B depicting aortic valve 12 in the valve-closed position.

FIG. 2A is a radial cross-sectional view depicting an exemplary embodiment of an inverted valve within the heart.

FIG. 2B is a radial cross-sectional view depicting an exemplary embodiment of the inverted valve during diastolic conditions.

FIGS. 3A-B are perspective views depicting an exemplary embodiment of the inverted valve in the valve-closed and valve-open positions, respectively.

FIGS. 3C-D are top down views depicting another exemplary embodiment of the inverted valve.

FIG. 4A is a perspective view depicting another exemplary embodiment of the inverted valve.

FIG. 4B is a top down view depicting another exemplary embodiment of the inverted valve.

FIG. 5A is a perspective view depicting an additional exemplary embodiment of the inverted valve.

FIG. 5B is a top down view depicting another exemplary embodiment of the inverted valve.

FIGS. 6A-C are perspective views of another exemplary embodiment of the inverted valve.

FIG. 6D is a top down view depicting another exemplary embodiment of the inverted valve.

### DETAILED DESCRIPTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which these inventions belong. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present inventions.

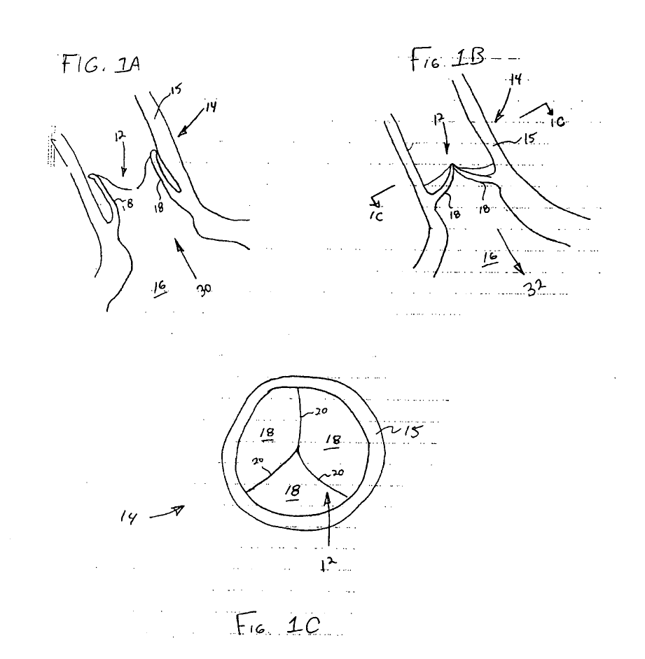

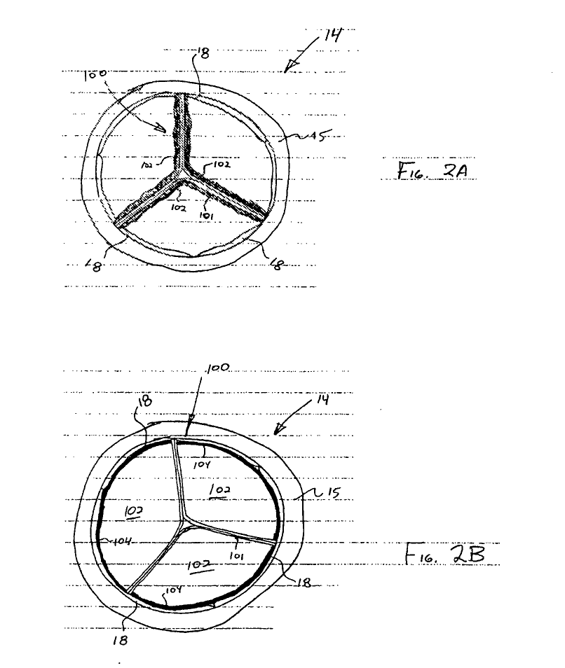

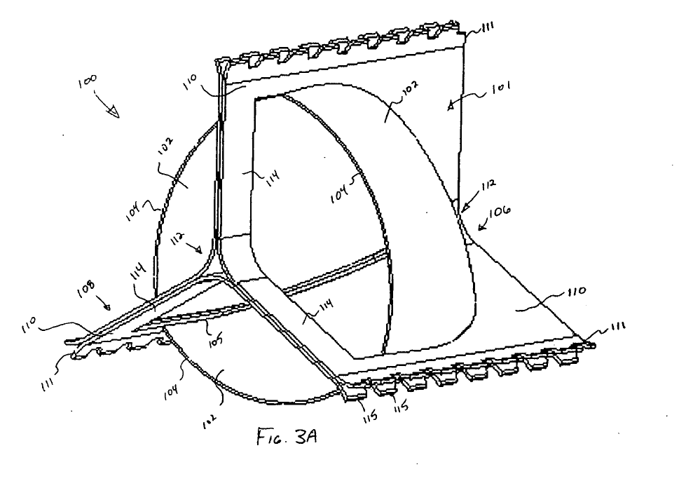

Provided herein are improved prosthetic valves and methods for implanting the same. These improved valves have non stent-like scaffolding structures that provide high radial stiffness along with crimpability and maximize fatigue life. The improved valves also have an inverted orientation that differs from the orientation of native valves like the aortic valve. FIG. 1A is a longitudinal cross-sectional view depicting a native aortic valve 12 within the heart. Aortic valve 12 is located within aorta 14 and borders left ventricle 16. Aortic valve 12 includes three natural leaflets 18 (only two are shown here). These leaflets 18 are depicted in the valve-open position during systolic conditions when the blood pressure gradient exists in directions 30. Under these conditions, each leaflet 18 is generally oriented against the aortic wall 15. FIG. 1B is another longitudinal cross-sectional view depicting leaflets 18 in the valve-closed position during diastolic conditions, where the blood pressure gradient exists in direction 323. Here, each leaflet 18 has deflected away from aortic wall 15 and into the path of blood flow. A commissure edge 20 along each leaflet 18 contacts similar commissure edges 20 on adjacent leaflets 18, creating a seal that prevents the backflow of blood into left ventricle 16, also referred to as regurgitation. FIG. 1C is a radial cross-sectional view taken along line 1C-1C of FIG. 1B depicting aortic valve 12 in the valve-closed position.

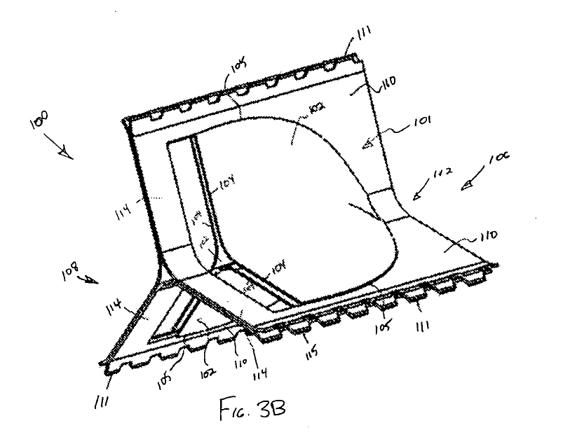

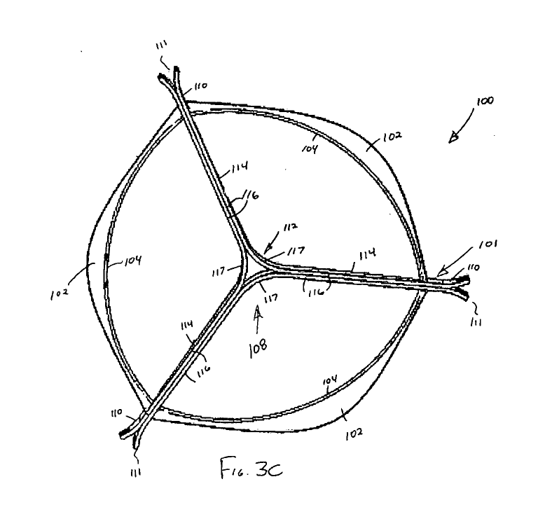

FIG. 2A is a radial cross-sectional view depicting an exemplary embodiment of an inverted valve 100 within the heart. Here, inverted valve 100 includes a support structure 101 and three valve leaflets 102. Leaflets 102 are depicted in the valve-open position during systolic conditions. Each leaflet is deflected toward the center of aorta 14 away from aortic wall 15. In this exemplary embodiment, support structure 101 has been delivered over native valve leaflets 18, which are held in the valve-open position by support structure 101. FIG. 2B is another radial cross-sectional view depicting leaflets 102 in the valve-closed position during diastolic conditions. Here, leaflets 102 have deflected towards the perimeter of aorta 14 and are preferably in contact with aortic wall 15 or native valve leaflets 18. Each leaflet 102 includes a free, commissure edge 104 that contacts the peripheral aortic tissue and creates a seal for preventing regurgitation. Commissure edge 104 of prosthetic leaflet 102 can also seal between the native leaflet 18 and/or the native aortic wall 15.

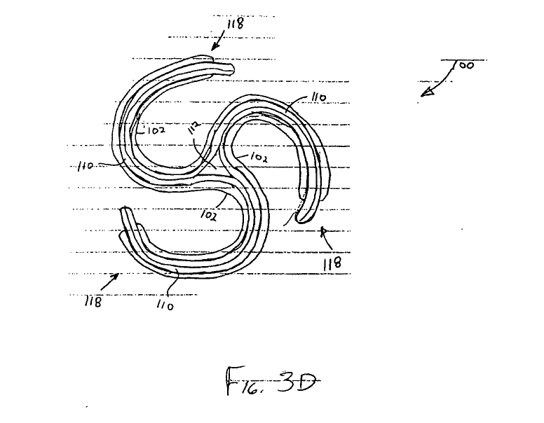

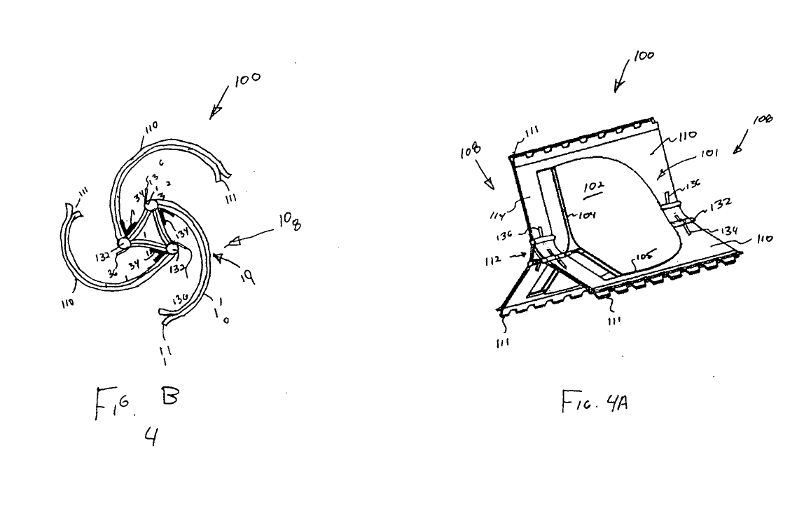

FIGS. 3A-B are perspective views depicting an exemplary embodiment of inverted valve 100 in the valve-closed and valve-open positions, respectively. Support structure 101 includes a first end 106 and a second end 108. First end 106 is generally oriented upstream from second end 108, with reference to the normal direction of blood flow under systologic conditions. Accordingly, end 106 will be referred to as “upstream” end 106 and end 108 will be referred to as “downstream” end 108.

Support structure 101 can include two or more strut sections 110 that meet and are coupled together at a central section 112. Here, support structure 101 includes three strut sections 110. Opposite central section 112 is an outer edge 111 of each strut 110, which is preferably configured to engage aortic wall or the native valve leaflets (not shown). Here, outer edge 111 includes a plurality of anchor devices 115 that are configured to anchor support structure 101 on aortic wall 15. Each strut 110 can include a reinforcement arm 114 located on downstream end 108 of support structure 101. Reinforcement arm 114 is preferably configured to provide additional reinforcement to support structure 101 before, during and after implantation.

Each strut 110 is preferably configured to allow movement of leaflets 102 between the valve-open and valve-closed positions. In this embodiment, each strut has an annular, or ring-like congfiguration with an open space located within. Each portion of strut 110 can be relatively planar, such as that depicted in FIGS. 3A-B, or can be rounded or any other desired rounded or polygonal shape, or combination thereof. As can be seen in FIG. 3A, when in the valve-closed position (stopping blood flow), commissure edges 104 form a generally ring-like profile, although other profiles can be used as desired, including circular, elliptical and irregular profiles. Preferably, the profile is sufficiently large to allow contact between commissure edges 104 and the adjacent aortic wall 15.

Although not shown here, commissure edges 104 can also be flared away from the center of valve 100. This increases the ability of each leaflet 102 to deflect to the valve-closed position as the blood pressure within the aorta exceeds that of that of the left ventricle. When deflected into the valve-closed position, the flared commissure edge 104 contacts the aortic wall or the native leaflet to a relatively greater degree, allowing for greater sealing effect.

In addition to a commissure edge 104, each leaflet 102 also preferably includes an attachment edge 105 for coupling to one or more struts 110. Attachment edge 105 of each leaflet 102 is preferably continuously attached to strut 110 along the length of the edge 105 to form an optimum seal. Leaflet 102 can be attached in any manner desired, including, but not limited to sewing, adhesives, clamping and the like.

In this embodiment, three leaflets 102 are attached to support structure 101 between adjacent struts 110. Each leaflet 102 can shift from the open to the closed position based on the blood pressure variations within the vasculature. As depicted in FIG. 3B, when in the valve-open position, each leaflet 102 preferably deflects towards the center of valve 100 with minimal folding or wrinkling, reducing the risk of damage to leaflet 102.

Support structure 101 can be configured from multiple pieces and joined into a common structure, or structure 101 can be a single monolithic construction. It should be noted that support structure 101 can also be formed from tubular material or can be wound or otherwise formed from a wire material. FIG. 3C is a top down depiction of downstream end 108 of support structure 101 with leaflets 102 shown in the valve-closed position. Here it can be seen that each strut 110 can be formed from two adjacent, panel-like members 116, which are preferably coupled together. Each member 116 can have a curved or bent portion 117 in it's mid-section in a location corresponding to central section 112. Each member 116 can be used to form one side of two adjacent struts 110. In this embodiment, curved portions 117 together define central section 112 of support structure 101, which can be configured to allow passage of a guidewire therethrough.

For instance, when housed within a lumen of the delivery device, it may be desirable to pass a guidewire through the lumen to aid in navigating the delivery device through the patient's vasculature. The guidewire can be routed through central sections 112 on upstream and downstream ends 106 and 108. To prevent blood leakage through central sections 112 after implantation, each central section 112 can be filled with a self-closing, compliant material configured to allow passage of the guidewire therethrough and to seal itself after removal of the guidewire. Alternatively, each leaflet 102 near upstream end 106 can be configured to conform around the guidewire during delivery and, upon removal of the guidewire, conform against adjacent leaflets 102 to provide a hemodynamic seal during diastolic conditions.

Valve 100 is preferably configured for percutaneous delivery through the vasculature of the subject. This delivery method can eliminate the need to use a blood-oxygenation machine (e.g., a heart and lung machine, cardio pulmonary bypass) and greatly reduces the risks associated with surgical valve replacement procedures (e.g., open heart surgery). Valve 100 can be placed in a contracted configuration to allow housing within a delivery device, such as a catheter and the like, percutaneous entry into the subject, such as through the femoral artery, and advancement through the vasculature into proximity with the valve to be replaced. Once positioned appropriately, valve 100 can be expanded into an expanded configuration for operation as a replacement valve.

Leaflets 102 preferably remain in a relatively flat, uncreased configuration while transitioning between the valve-open and valve-closed configurations. Folding and creasing of the prosthetic tissue leaflets 102 is preferably avoided to reduce the risk of mechanically damaging leaflets 102. Folds, creases and other manipulations of leaflets 102 can contribute to reduced valve life due to fatigue, as well as being a nidus for calcification.

The valve support structure 101 is preferably configured to minimize contact between leafelts 102 and support structure 101. For instance, in the embodiments described with respect to FIGS. 3A-D, struts 110 are configured in a ring-like shape and support leaflets 102 within the central open space to minimize the contact of prosthetic leaflet 102 with support structure 101 during the cardiac cycle. This reduces wear on prosthetic leaflet 102 and can increase valve life and durability.

FIG. 3D is a top down view depicting an exemplary embodiment of valve 100 in the contracted configuration. Here, each strut 110 has been curled, or rolled to form a multi-lobe structure. To form this multi-lobe structure, each of the three struts 110 is rotated toward the center longitudinal axis of valve 100 into a lobe 118. The multi-lobed structure has a reduced cross-sectional profile that will allow valve 100 to be housed within the delivery device. After being placed into proximity with the desired valve implantation site, struts 110 can be uncurled to the expanded, relatively straightened state.

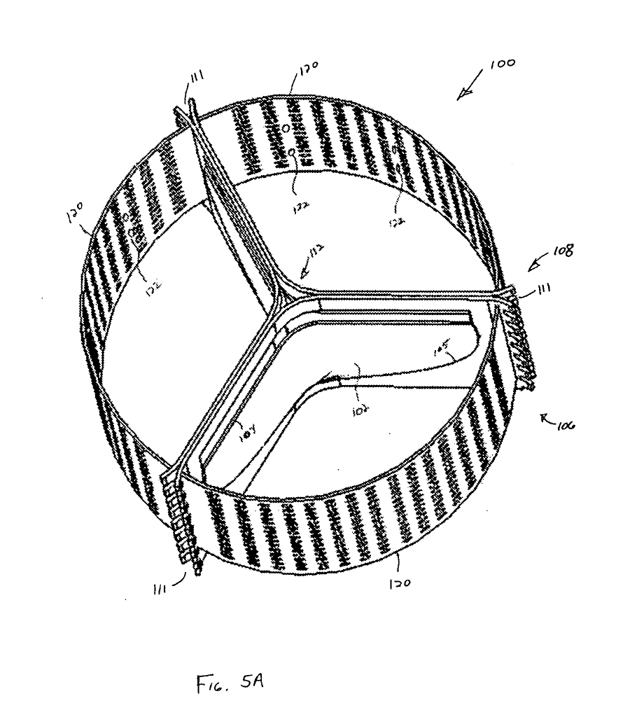

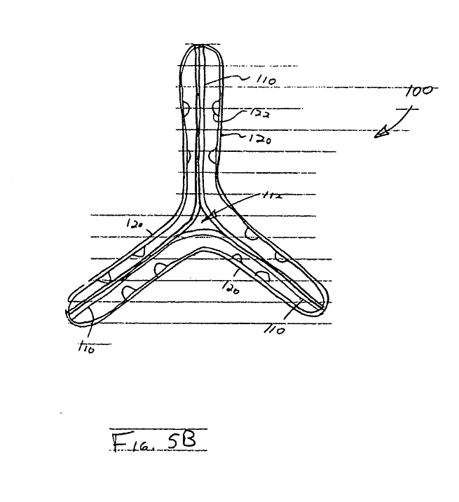

FIG. 4A is a perspective view of another exemplary embodiment of valve 100. In this embodiment, each strut 110 is coupled to central section 112 by way of a hinge 132. Hinge 132 facilitates the transition of struts 110 between the relatively straightened state shown here, and the curled state of the multi-lobe configuration depicted in the top down view of FIG. 4B. A bias member 134 is coupled between each strut 110 and central strut 130 to facilitate the transition from the multi-lobe configuration to the expanded configuration. Bias member 134 applies a bias to deflect each strut 110 away from central strut 130 and into the orientation of the relatively straightened state, where each strut 110 is oriented approximately 120 degrees apart. Bias member 134 can be any member configured to apply a bias and can be coupled with support structure 110 in any manner suitable for implantable medical devices. Here, bias member 134 is configured as a spring and is joined to support structure 110.

To prevent travel of strut 110 significantly past the 120 degree orientation described with respect to FIG. 4B, a counteraction member 136 can be included. Here, counteraction member 136 is configured as an abutment on the opposite side of strut 110 from bias member 134. Counteraction member 136 can be also be configured as a second, opposing bias element.

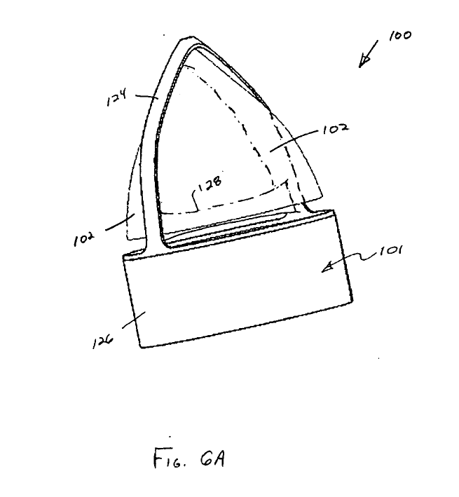

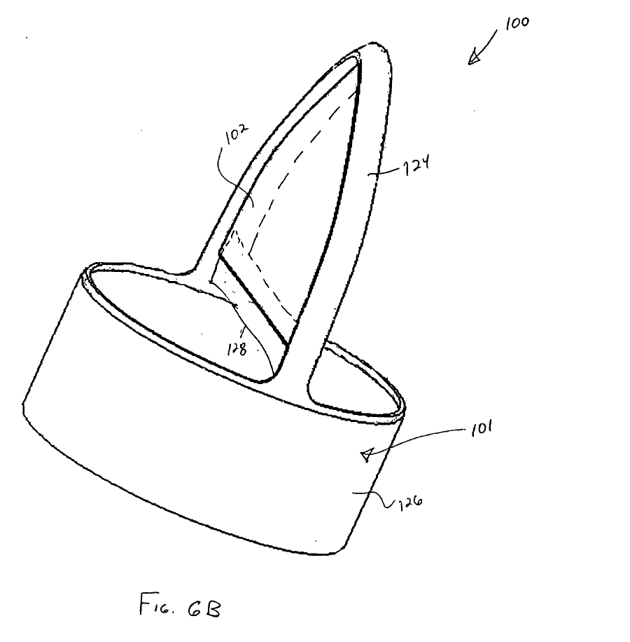

FIG. 5A is a perspective view depicting another exemplary embodiment of the valve 100. Here, support structure 101 includes invertable panels 120, such as those described in U.S. published patent application 2005/0203614, entitled “Prosthetic Heart Valves, Scaffolding Structures, and Methods of Implantation of Same,” copending U.S. patent application Ser. No. 11/425,361, entitled “Prosthetic Heart Valves, Support Structures And Systems And Methods For Implanting The Same,” and copending U.S. provisional patent application Ser. No. 60/805,329, entitled “Prosthetic Heart Valves, Support Structures And Systems And Methods For Implanting The Same,” each of which is fully incorporated by reference herein.

FIG. 5B is a top down view depicting this embodiment after panels 120 have been inverted. Here, panels 120 lie adjacent to struts 110 with valve leaflets 102 located therebetween. This configuration has been referred to as a three-vertex star-shaped structure in the 2005/0203614 application. Panels 120 and struts 110 can then be rolled or curved into the multi-lobe configuration, similar to that described with respect to FIG. 3D.

Panels 120 and/or struts 110 can include one or more spacers 122 to space the distance between panels 120 and struts 110 after panels 120 have been inverted. The spaced distance is preferably on the order of the thickness of valve leaflets 102 or greater, in order to avoid compression of valve leaflet 102. If desired, spacers 122 can be placed at locations on panel 120 and/or strut 110 where no valve leaflet is present, to avoid contact with valve leaflet 102. Alternatively, spacers 122 can be placed opposite valve leaflet 102 and configured to contact valve leaflet 102, preferably in a manner that minimizes the risk of mechanically damaging valve leaflet 102. Spacers 122 can be formed in the surface of panels 120 and/or struts 110, or can be attached thereto. If attached, spacers 122 can be formed from a separate material including soft, pliable, biocompatible materials such as polymers, natural tissues, and the like.

To facilitate the inversion of panels 120, as well as the curling of panels 120 and struts 110, support structure 101 is preferably formed from a biocompatible, elastic material such as NITINOL, elgiloy, stainless steel, polymers and the like. The selected material is preferably biased towards the desired fully deployed shape. For instance, panels 120 are preferably biased towards the expanded configuration described with respect to FIG. 5A, and struts 110 are preferably biased towards the expanded configuration described with respect to FIGS. 3A-D and 5A. This can he accomplished using any technique known in the art, such as by the heat treatment of NITINOL. Panels 120 can be configured in numerous different manners, including those described in each of the incorporated references, depending on the desired functionality. For instance, each panel 120 can be formed as a separate structure independent from the other elements, or panels 120 can be formed as regions within one continuous annular structure.

The multi-lobed structures described herein are similar to the multi-lobed structure described with respect to FIG. 2C of the incorporated U.S. published patent application 2005/0203614. Exemplary embodiments of delivery devices for converting valve 100 between a multi-star structure and a multi-lobed structure and delivering valve 100 to the implantation site are also described therein. For instance, one exemplary embodiment of a delivery device suitable for use with valve 100 is described with respect to FIGS. 12A-F of the incorporated application. Additional types of delivery devices usable with valve 100 are also described in copending U.S. patent application Ser. No. 11/364,715, entitled “Methods And Devices For Delivery Of Prosthetic Heart Valves And Other Prosthetics,” which is fully incorporated by reference herein.

While the embodiment of valve 100 described above with respect to FIGS. 3A-D does not include invertable panels 120 as do some of the other embodiments described in the incorporated documents, one of ordinary skill in the art will readily recognize that the added functionality of the delivery devices used to invert and expand the panels can be omitted.

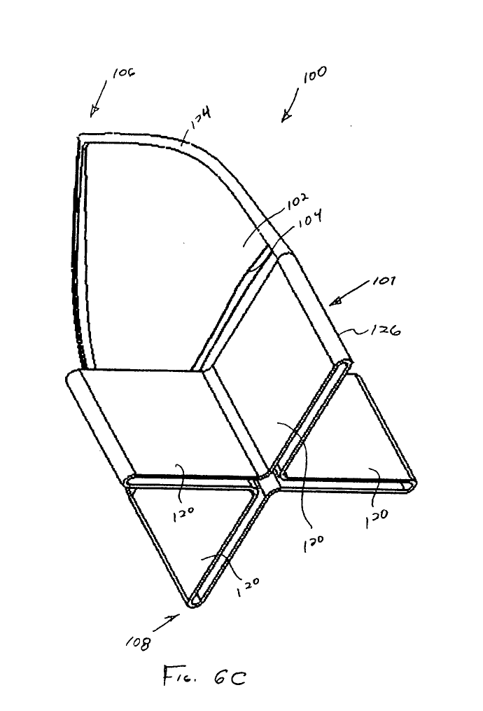

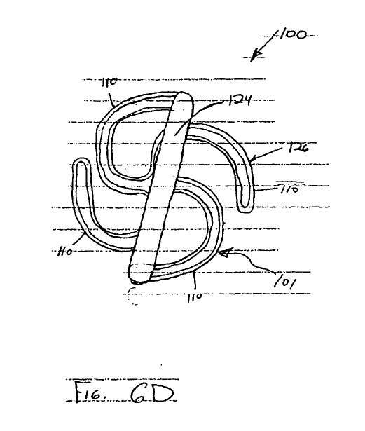

FIGS. 6A-B are perspective views of another exemplary embodiment of inverted valve 100. In FIG. 6A, valve leaflets 102 are shown in the valve closed position, while in FIG. 6B, valve leaflets 102 are shown in the valve open position. In this embodiment, support structure 101 includes an elongate, curved support arm 124 coupled with an anchor section 126. Two valve leaflets 102 are preferably coupled with support arm 124, which, in turn, can be anchored against aortic wall 15 (not shown) by anchor section 126. Valve leaflets 102 are coupled on either side of a guide structure 128, which can ensure that leaflets 102 deflect in the proper direction to close the valve. Because it lies in the blood stream, the width of support arm 124 is preferably minimized to lessen any hemodynamic effects.

In this embodiment, anchor section 126 includes four invertable panels 120. FIG. 6C is a bottom up view depicting this exemplary embodiment of valve 100 with panels 120 in the inverted state. FIG. 6D is a top down view depicting this exemplary embodiment of valve 100 in the multi-lobe configuration. Support arm 124 and guide structure 128 are preferably configured to bend and/or twist to accommodate entry into this multi-lobe configuration.

One of the many advantages the embodiments described herein have over conventional designs is the ability to open and close the valve without the use of moving mechanical parts, outside of leaflets 102. For instance, in some conventional designs, the valve opens and closes like an umbrella, with numerous joints pivotally fixed to mechanical arms that oscillate back and forth with each valve cycle to open and close the umbrella-like valve. These additional mechanical components and joints add complexity and increases the risk of premature device failure. In the embodiments described herein, the support structure is static and capable of operating without this added mechanical complexity.

The preferred embodiments of the inventions that are the subject of this application are described above in detail for the purpose of setting forth a complete disclosure and for the sake of explanation and clarity. Those skilled in the art will envision other modifications within the scope and spirit of the present disclosure. Such alternatives, additions, modifications, and improvements may be made without departing from the scope of the present inventions, which is defined by the claims.

What is claimed is:

1. A method for implanting a prosthetic valve, comprising:
placing an implantable valve in proximity with an aortic valve implantation site of a subject;
engaging the valve with the implantation site, wherein the valve comprises a support structure and at least two valve leaflets coupled with the support structure, the valve leaflets configured to deflect towards the aortic wall into a first position for resisting the flow of blood towards the left ventricle and configured to deflect away from a aortic wall into a second position for allowing the flow of blood from a left ventricle, wherein the support structure is configured to remain static during deflection of the valve leaflets.

2. The method of claim 1, wherein the support structure comprises a plurality of struts, each strut configured to support at least one valve leaflet.

3. The method of claim 2, wherein engaging the valve with the implantation site comprises transitioning the struts from a curled state to a relatively straightened state configured to engage tissue.

4. The method of claim 3, wherein the struts are biased towards the straightened state.

5. The method of claim 4, wherein the support structure further comprises a bias member coupled to each strut.

6. The method of claim 1, wherein the support structure comprises a support arm coupled with an anchor section, the support arm being coupled with at least two valve leaflets.

7. The method of claim 6, wherein the anchor section comprises a plurality of invertable panels.

8. The method of claim 2, wherein each strut comprises a plurality of anchor devices configured to anchor the valve at the implantation site.

9. The method of claim 2, wherein each strut comprises a first edge coupled with one or more valve leaflets.

10. The method of claim 9, wherein each strut comprises an aperture configured to receive one or more valve leaflets in the second position.

11. The method of claim 2, wherein the support structure comprises three struts.

12. The method of claim 11, wherein each strut is oriented generally 120 degrees about a longitudinal center axis of the support structure.

13. The method of claim 12, wherein each strut is coupled with a central section of the support structure.

14. The method of claim 13, wherein each strut is coupled with the central section of the support structure by way of a hinge.

15. The method of claim 14, wherein engaging the valve with the implantation site comprises rotating each strut about the hinge from a relatively curled state to a relatively straightened state.

16. A prosthetic valve apparatus, comprising:
a support structure configured for implantation within a blood vessel; and
at least two valve leaflets coupled with the support structure, the valve leaflets configured to deflect towards the aortic wall into a first position for resisting the flow of blood towards a heart chamber and configured to deflect away from a wall of the vessel into a second position for allowing the flow of blood from the heart chamber, wherein the support structure is configured to remain static during deflection of the valve leaflets.

17. The apparatus of claim 16, wherein the support structure comprises a plurality of struts, each strut configured to support at least one valve leaflet.

18. The apparatus of claim 17, wherein the struts are configured to transition from a curled state to a relatively straightened state configured to engage tissue.

19. The apparatus of claim 18, wherein the struts are biased towards the straightened state.

20. The apparatus of claim 19, wherein the support structure further comprises a bias member coupled to each strut.

21. The apparatus of claim 20, wherein the support structure comprises a support arm coupled with an anchor section, the support arm being coupled with at least two valve leaflets.

22. The apparatus of claim 21, wherein the anchor section comprises a plurality of invertable panels.

23. The apparatus of claim 17, wherein each strut comprises a plurality of anchor devices configured to anchor the valve at the implantation site.

24. The apparatus of claim 17, wherein each strut comprises a first edge coupled with one or more valve leaflets.

25. The apparatus of claim 24, wherein each strut comprises an aperture configured to receive one or more valve leaflets in the second position.

26. The apparatus of claim 17, wherein the support structure comprises three struts.

27. The apparatus of claim 26, wherein each strut is oriented generally 120 degrees about a longitudinal center axis of the support structure.

28. The apparatus of claim 27, wherein each strut is coupled with a central section of the support structure.

29. The apparatus of claim 28, wherein each strut is coupled with the central section of the support structure by way of a hinge.

30. The apparatus of claim 29, wherein each strut is rotatable about the hinge from a relatively curled state to a relatively straightened state.

31. The apparatus of claim 2, wherein the support structure comprises:
a central section coupled with three strut sections, each strut section having a free edge configured to engage the wall of the blood vessel, wherein the strut sections comprise an inner edge defining an open space configured to allow deflection of the valve leaflets, the inner edge being coupled with the plurality of valve leaflets.

32. The apparatus of claim 31, wherein each of the strut sections are oriented symmetrically.

33. The apparatus of claim 31, wherein each of the strut sections are oriented approximately 120 degrees apart about the central section.

34. The apparatus of claim 31, wherein the support structure is collapsible for implantation via an elongate medical device.

35. The apparatus of claim 2, wherein the support structure comprises:
an anchor section configured to engage the wall of the blood vessel; and
a support arm comprising two ends, each end being coupled with an opposite side of the anchor section such that the support arm extends across the anchor section, wherein the support arm is coupled with the plurality of valve leaflets and configured to allow deflection of the valve leaflets.

36. The apparatus of claim 35, wherein the support arm is curved.

37. The apparatus of claim 36, wherein the support arm is coupled with a guide structure configured to guide deflection of the valve leaflets.

38. The apparatus of claim 37, wherein the guide structure is a planar structure.

39. The apparatus of claim 36, wherein the anchor section has circular radial cross-section.
